(19) United States
(12) Patent Application Publication
(10) Pub. No.: US 2026/0199608 A1
(43) Pub. Date: Jul. 16, 2026

(54) A SUB-ASSEMBLY OF A MEDICAMENT DELIVERY DEVICE

(71) Applicant: SHL Medical AG, Zug (CH)

(72) Inventors: Thad Miley, Deerfield Beach, FL (US); Rayyan Naji, Deerfield Beach, FL (US)

(21) Appl. No.: 19/136,380

(22) PCT Filed: Dec. 1, 2023
(86) PCT No.: PCT/EP2023/083889

§ 371 Date: Jun. 6, 2025

Related U.S. Application Data
(60) Provisional application No. 63/432,725, filed on Dec. 15, 2022.
(60) Provisional application No. 63/444,284, filed on Feb. 9, 2023.

(30) Foreign Application Priority Data
Mar. 14, 2023 (EP) 23161845.5

(51) Int. Cl. A61M 5/32; A61M 5/20

(52) U.S. Cl. CPC A61M 5/3257; A61M 5/2033

(57) ABSTRACT

The present disclosure provides a sub-assembly of a medicament delivery device including a housing extending along a longitudinal axis between a proximal end and a distal end. The housing is configured to accommodate a medicament container. The medicament container is coupled to a medicament delivery member comprising an outer part for delivering medicament. The outer part of the medicament delivery member is configured to extend beyond the proximal end of the housing in the direction of the longitudinal axis. The sub-assembly further includes a delivery member guard partially arranged within the housing. The delivery member guard is telescopic relative to the proximal end of the housing. The delivery member guard is movable along the longitudinal axis between an extended position where the delivery member guard is configured to fully surround the outer part of the medicament delivery member of the medicament container and a retracted position where the delivery member guard is configured to partially surround the outer part of the medicament delivery member of the medicament container.

### TECHNICAL FIELD

The present disclosure generally relates to a sub-assembly of the medicament delivery device, particularly to a sub-assembly of the medicament delivery device comprising a medicament delivery guard.

### BACKGROUND

Medicament delivery devices such as auto-injectors, inhalers, or on-body devices are generally known for the self-administration of a medicament by patients without formal medical training. For example, patients suffering from diabetes or people who are undergoing an artificial fertilization procedure may require repeated injections of insulin or hormone. Other patients may require regular injections of other types of medicaments, such as a growth hormone. Therefore, medicament delivery devices for self-administration usually are arranged with multiple automatic functions and protection features. For example, a medicament delivery member guard is commonly used. The medicament delivery member guard is configured to cover a medicament delivery member, e.g., an injection needle, so that a user will not accidentally be in contact with the medicament delivery member.

The medicament delivery member guard is usually arranged to be telescopically movable relative to the housing of the medicament delivery device. The medicament delivery member guard is configured to be in contact with the medicament delivery site. A medicament delivery device that is configured to be triggered, e.g., inserting the injection needle into the medicament delivery site and/or expelling contained medicament to the medicament delivery site, by the movement of the medicament delivery member guard, for example, the medicament delivery device is triggered once the medicament delivery member guard is fully pressed against the medicament delivery site, or the medicament delivery device is triggered when the medicament delivery member guard is fully pressed against the medicament delivery site and a button is manually pressed by the user is a preferred solution in the market. However, there are still rooms to improve the such design.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device comprising a housing extending along a longitudinal axis between a proximal end and a distal end; wherein the housing is configured to accommodate a medicament container; wherein the medicament container is coupled to a medicament delivery member comprising an outer part for delivering medicament; wherein the outer part of the medicament delivery member is configured to extend beyond the proximal end of the housing in the direction of the longitudinal axis; a delivery member guard partially arranged within the housing; wherein the delivery member guard is telescopic relative to the proximal end of the housing; wherein the delivery member guard is movable along the longitudinal axis between an extended position where the delivery member guard is configured to fully surround the outer part of the medicament delivery member of the medicament container and a retracted position where the delivery member guard is configured to partially surround the outer part of the medicament delivery member of the medicament container; and wherein a proximal end of the delivery member guard is configured to extend beyond the proximal end of the housing when the delivery member guard is in the retracted position.

Preferably, according to another embodiment, when the delivery member guard is in the retracted position, a length measured along the longitudinal axis from the proximal end of the delivery member guard to the proximal end of the housing is at least 2 mm.

Preferably, according to another embodiment, when the delivery member guard is in the retracted position, the length measured along the longitudinal axis from the proximal end of the delivery member guard to the proximal end of the housing is between 3 mm to 6 mm.

Preferably, according to another embodiment, when the delivery member guard is in the retracted position, the length measured along the longitudinal axis from the proximal end of the delivery member guard to the proximal end of the housing is 3 mm, 3.3 mm, or 3.9 mm.

Preferably, according to another embodiment, the contact section comprises a contact surface perpendicular to the longitudinal axis and a neighbouring sleeve extending from the contact surface in the direction of the longitudinal axis towards the distal end of the housing.

Preferably, according to another embodiment, the delivery member guard comprises a contact section configured to be in contact with a medicament delivery site.

Preferably, according to another embodiment, the contact section comprises a contact surface perpendicular to the longitudinal axis and a neighbouring sleeve extending from the contact surface in the direction of the longitudinal axis towards the distal end of the housing.

Preferably, according to another embodiment, the contact surface extends beyond the neighbouring sleeve in the direction transverse to the longitudinal axis.

Preferably, according to another embodiment, the delivery member guard comprises a proximal tubular section extending beyond the proximal end of the housing when the delivery member guard is in the extended position.

Preferably, according to another embodiment, a length of the proximal tubular section measured along the longitudinal axis is between 14 mm to 18 mm from the proximal end of the delivery member guard 2 to the proximal end of the housing.

Preferably, according to another embodiment, the sub-assembly of the medicament delivery device comprises a drive mechanism comprising a power source.

Preferably, according to another embodiment, when the power source is released, the power source is configured to act on the medicament container and force a contained medicament to be expelled via the medicament delivery member.

Preferably, according to another embodiment, the drive mechanism is arranged within the housing.

Preferably, according to another embodiment, the power source can be released only when the delivery member guard is in the retracted position.

Preferably, according to another embodiment, the power source is configured to be released by the delivery member guard when the delivery member guard is moved to the retracted position.

Preferably, according to another embodiment, the sub-assembly of the medicament delivery device comprises a button extending out of the housing.

Preferably, according to another embodiment, the power source is configured to be released when the button is moved towards the housing and the delivery member guard is in the retracted position.

Preferably, according to another embodiment, the drive mechanism comprises a plunger rod configured to be moved into the medicament container.

Preferably, according to another embodiment, the power source is a spring engaged with the plunger rod.

Preferably, according to another embodiment, the spring is configured to move the plunger rod into the medicament container when the spring is released.

Preferably, according to another embodiment, the drive mechanism comprises a rotator rotatable around the longitudinal axis between a first position where the rotator is coupled to the plunger rod such that the plunger rod is axially immovable relative to the rotator and a second position where the rotator is decoupled from the plunger rod such that the plunger rod is axially moved by the power source.

Preferably, according to another embodiment, the rotator comprises a chamfered surface.

Preferably, according to another embodiment, the delivery member guard comprises an interacting surface configured to be moved along the chamfer surface of the rotator when the delivery member guard is moved from the extended position to the retracted position such that the rotator is moved from the first position to the second position.

Preferably, according to another embodiment, the drive mechanism comprises an actuator sleeve axially movable relative to the housing between a proximal position where the actuator sleeve is coupled to the plunger rod such that the plunger rod is axially immovable relative to the actuator sleeve and a second position where the actuator sleeve is decoupled from the plunger rod such that the plunger rod is axially moved by the power source.

Preferably, according to another embodiment, the actuator sleeve comprises a proximally directed surface engaged with a distally directed surface of the delivery member guard such that when the delivery member guard is moved from the extended position to the retracted position the actuator sleeve is moved from the first position to the second position by the delivery member guard.

Preferably, according to another embodiment, the delivery member guard is biased by a biasing member to the proximal end of the housing.

Preferably, according to another embodiment, the biasing member is a spring.

The sub-assembly can be used in a medicament delivery device that is an injection device, an inhalation device, or a medical sprayer.

Preferably, according to another embodiment, the medicament delivery device is an auto-injector.

Preferably, according to another embodiment, the medicament delivery device is a hand-held, pen-type auto-injector.

Preferably, according to another embodiment, the medicament delivery member is an injection needle or a spray nozzle.

Preferably, according to another embodiment, the medicament delivery comprises a medicament delivery member, and the medicament delivery member is an injection needle.

Preferably, according to another embodiment, a length measured along the longitudinal axis L from a proximal tip end of the injection needle to the proximal end of the delivery member guard when the delivery member guard is in the retracted position is 4 mm to 12 mm; preferably, is 6 mm, 8 mm, or 10 mm.

Preferably, according to another embodiment, the medicament container of the medicament delivery device is a syringe, a cartridge or a collapsible bag.

Preferably, according to another embodiment, the medicament container of the medicament delivery device is made of glass material or plastic material.

Preferably, according to another embodiment, the medicament container of the medicament delivery device comprises a single chamber containing a single type of medicament substance.

Alternatively, according to another embodiment, the medicament container of the medicament delivery device comprises multiple chambers containing multiple substances respectively.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example, pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### DETAILED DESCRIPTION

A medicament delivery device usually comprises a housing for accommodating a medicament container containing prefilled medicament. The housing is commonly tubular. A delivery member guard is commonly used in the medicament delivery device. The delivery member guard is configured to cover a medicament delivery member, e.g., an injection needle, so that a user will not accidentally be in contact with the medicament delivery member.

The delivery member guard is usually arranged to be telescopically movable relative to the housing of the medicament delivery device. The delivery member guard is configured to be in contact with the medicament delivery site. A medicament delivery device that is configured to be triggered, e.g., automatically inserting the injection needle into the medicament delivery site and/or expelling contained medicament to the medicament delivery site, by the movement of the delivery member guard, for example, the medicament delivery device is triggered once the medicament delivery member guard is fully pressed against the medicament delivery site or is pressed to move with a certain distance (the expression of ‘the medicament delivery member guard is fully pressed ’ means that the medicament delivery member guard cannot be moved any further towards the distal end of the housing of the medicament delivery device), or the medicament delivery device is triggered when the delivery member guard is fully pressed against the medicament delivery site or is pressed to move with a certain distance and a button is manually pressed by the user is a preferred solution in the market. However, in some cases, users believe they have followed an instruction to operate the medicament delivery device correctly, but the device does not be triggered on the first attempt, or a perceived large force is required to trigger the medicament delivery device. One of the reasons might be an obstacle to the movement of the delivery member guard, e.g., too much skin, as the obstacle, contact with a housing of the medicament delivery device so that the delivery member guard cannot or is difficult to be moved to a trigger position (a position that the medicament delivery device will be triggered or will be triggered once the button is pressed). As a result, users cannot complete a trigger action, or the trigger force is high. One of the reasons for this issue might be that the delivery member guard is designed to be moved closer to the housing, for example, a length X2 measured along the longitudinal axis L from a proximal end of the delivery member guard and a proximal end of the housing when the delivery member guard is pressed to trigger the medicament delivery action is generally about 1 mm or less, thus, there is a risk that the skin around the medicament delivery site might be trapped between the proximal end of the delivery member guard and the proximal end of the housing. In one preferred example, the delivery member guard is configured to be moved 10 mm to 15 mm in the distal direction relative to the housing into the trigger position. Preferably, for subcutaneous injection, the delivery member guard is configured to be moved 12.05 mm+/−10% dependent on the engineering tolerance in the distal direction relative to the housing into the trigger position. It should be noted that the distance can be modified for different needle lengths or predetermined injection depths.

It should be noted that most of the medicament delivery devices are designed with a bandwidth for the trigger movement; meaning that instead of designing the medicament delivery device to be triggered at the point that the medicament delivery member guard is fully pressed against the medicament delivery site, the medicament delivery devices usually are designed to be triggered when the medicament delivery member guard is moved with a certain range of distance. For example, if the medicament delivery member guard is designed to reach the ‘fully pressed’ position by moving 13 mm towards a housing of the medicament delivery device, the medicament delivery device usually will be designed to be triggered when the medicament delivery member guard is moved 12 mm to 13 mm towards a housing of the medicament delivery device to solve any potential engineering tolerance issue.

A preferred solution is to keep the housing away from the medicament delivery site during the trigger action (the expression of ‘trigger action’ means the user manually moves the delivery member guard towards the distal end of the housing to the trigger position of the delivery member guard) and reduce the friction force between the delivery member guard, skin, and housing. A more preferred solution is to keep the housing away from the medicament delivery site greater than 2 mm during the trigger action.

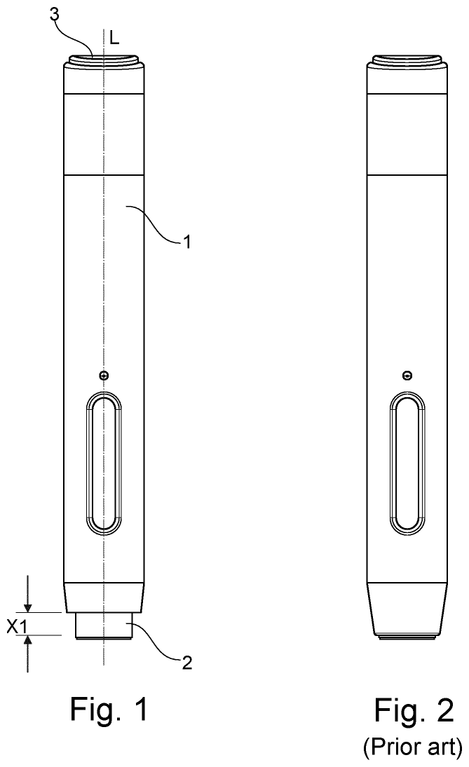

FIG. 1 and FIGS. 4A-4B show a sub-assembly of the medicament delivery device of the invention. The sub-assembly of the medicament delivery device comprises a housing 1; 1′ extending along a longitudinal axis L between a proximal end and a distal end. The housing 1 is configured to accommodate a medicament container. In a preferred example, the housing 1 is tubular. The medicament container is coupled to a medicament delivery member N comprising an outer part for delivering the medicament. The outer part of the medicament delivery member is configured to extend beyond the proximal end of the housing in the direction of the longitudinal axis for being in contact with a medicament delivery site so that the medicament contained within the medicament container can be delivered via the outer part of the medicament delivery member. In a preferred example, the medicament delivery member is an injection needle N, as shown in FIGS. 4A-B. In this example, the outer part of the medicament delivery member is the part of the injection needle that is configured to be exposed to the user and at least partially inserted into the injection site. A length of a proximal part of the injection needle that is measured along the longitudinal axis from a proximal end of the medicament container, e.g., a syringe, is dependent on the designed injection. For example, the length of the proximal part of the injection needle is commonly between 12.7 mm to 15.875 mm for the subcutaneous injection; and is commonly between 25 mm to 38 mm for the intramuscular injection. As the outer part of the injection needle extends beyond the proximal end of the housing 1, an effective injection depth is provided by the outer part of the injection needle. A length of the outer part of the injection needle is measured along the longitudinal axis L from a proximal tip end of the injection needle to the proximal end of the housing. The length of the proximal part of the injection needle defines the maximum length of the outer part of the injection needle.

The sub-assembly of the medicament delivery device comprises a delivery member guard 2; 2′ partially arranged within the housing 1; 1′. The delivery member guard 2; 2′ is telescopic relative to the proximal end of the housing 1; 1′. The delivery member guard 2; 2′ is movable along the longitudinal axis L between an extended position (as shown in FIG. 4A) where the delivery member guard 2; 2′ is configured to fully surround the outer part of the medicament delivery member N of the medicament container and a retracted position (as shown in FIG. 1 and FIG. 4B) where the delivery member guard 2; 2′ is configured to partially surround the outer part of the medicament delivery member N of the medicament container. The delivery member guard 2′ comprises a proximal tubular section 20 configured to fully surround the outer part of the medicament delivery member N of the medicament container when the delivery member guard 2′ is in the extended position and is configured to partially surround the outer part of the medicament delivery member N of the medicament container when the delivery member guard 2′ is in the retracted position. In a preferred example the proximal tubular section 20 of the delivery member guard 2′ is coaxial to the housing 1′.

A proximal end of the delivery member guard 2; 2′ is configured to extend beyond the proximal end of the housing 1; 1′ when the delivery member guard 2; 2′ is in the retracted position. In a preferred example, the proximal tubular section 20 of the delivery member guard 2 extends from the proximal end of the delivery member guard 2 to the proximal end of the housing 1 when the delivery member guard 2 is in the extended position.

In one example where the medicament delivery member N is an injection needle N, the delivery member guard 2; 2′ is configured to fully surround the outer part of the injection needle N of the medicament container when the delivery member guard 2; 2′ is in the extended position and is configured to partially surround the outer part of the medicament delivery member N of the medicament container when the delivery member guard 2; 2′ is in the retracted position.

The effective injection depth is defined by a length X3 measured along the longitudinal axis L from the proximal tip end of the injection needle N to the proximal end of the delivery member guard 2′ when the delivery member guard 2′ is in the retracted position. In a preferred example, the effective injection depth is 4 mm to 12 mm; preferably, is 6 mm, 8 mm, or 10 mm.

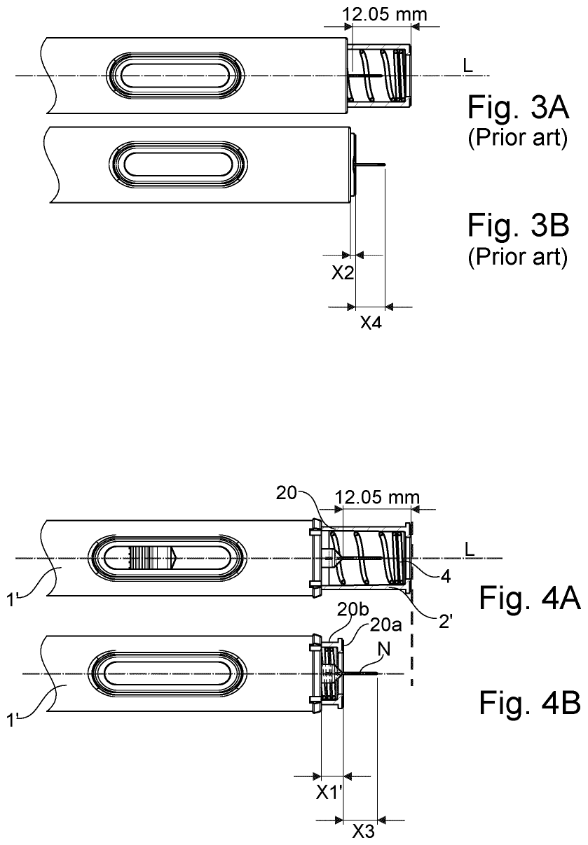

In one example, the housing 1; 1′ is shortened so that more parts of the delivery member guard 2; 2′ are exposed from the proximal end of the housing 1; 1′. Alternatively, in another example, the delivery member guard is elongated so that more parts of the delivery member guard are exposed from the proximal end of the housing. Therefore, the distance between the proximal end of the delivery member guard and the proximal end of the housing can be extended when the delivery member guard is in the retracted position, as shown in FIGS. 1-2 and FIGS. 3B, 4B, the skin contact area of the housing can be reduced. As a result, the design can reduce the force to trigger the sub-assembly of the medicament delivery device by moving the delivery member guard from the extended position to the retracted position. In one preferred example, the housing 1; 1′ is configured to be spaced apart from the medicament delivery site greater than 2 mm during the trigger action; meaning when the delivery member guard 2; 2′ is in the retracted position, a length X1 measured along the longitudinal axis L from the proximal end of the delivery member guard 2; 2′ to the proximal end of the housing 1; 1′ is at least 2 mm.

It should be noted that as the proximal tubular section 20 of the delivery member guard 2′ is configured to surround the outer part of the injection needle N of the medicament container so that the user will not accidentally contact the injection needle, thus, if the length X1 measured along the longitudinal axis L from the proximal end of the delivery member guard 2; 2′ to the proximal end of the housing 1; 1′ is long when the delivery member guard 2; 2′ is in the retracted position, the proximal tubular section 20 will also need to be long, thus, the material cost of medicament delivery devices might increase. Therefore, in a preferred example, the length X1 measured along the longitudinal axis L from the proximal end of the delivery member guard 2 to the proximal end of the housing 1 is between 3 mm to 6 mm when the delivery member guard is in the retracted position. In a preferred example, the length X1 measured along the longitudinal axis L from the proximal end of the delivery member guard 2 to the proximal end of the housing 1 is 3 mm, 3.3 mm, or 3.9 mm when the delivery member guard 2; 2′ is in the retracted position.

As the proximal end of the delivery member guard 2; 2′ is configured to be in contact with a medicament delivery site, the sub-assembly as disclosed above provides a length X1 measured along the longitudinal axis L from the proximal end of the delivery member guard 2; 2′ to the proximal end of the housing 1; 1′ being greater than 2 mm can keep the housing 1; 1′ away from the medicament delivery site during the trigger action and reduce the friction force between the delivery member guard 2; 2′, the skin, and the housing 1; 1′.

In one example, the delivery member guard 2′ comprises a contact section 20a, 20b configured to be in contact with a medicament delivery site. In another example, the contact section 20a, 20b is spaced apart from the proximal end of the housing 1′. In one example, the contact section 20a, 20b comprises a contact surface 20a perpendicular to the longitudinal axis L and a neighbouring sleeve 20b extending from the contact surface 20a in the direction of the longitudinal axis L towards the distal end of the housing 1′. In one example, the contact surface 20a is the proximal end of the delivery member guard 2′. In a preferred example, the contact surface 20a extends beyond the neighbouring sleeve 20b in the direction transverse to the longitudinal axis L, as shown in FIGS. 4A-4B. The contact surface 20a is configured to block skin tissue from being trapped between the housing 1′ and the delivery member guard 2′.

Furthermore, as mentioned above, the effective injection depth is defined by the length X3 measured along the longitudinal axis L from the proximal tip end of the injection needle N to the proximal end of the delivery member guard 2′ when the delivery member guard 2′ is in the retracted position. Thus, in a preferred example, a length of the proximal tubular section 20 of the delivery member guard 2′ is greater than the effective injection depth plus the length X1 measured along the longitudinal axis L from the proximal end of the delivery member guard 2 to the proximal end of the housing 1. In one preferred example, the length of the proximal tubular section 20 of the delivery member guard 2′ measured along the longitudinal axis L is between 14 mm to 18 mm measured along the longitudinal axis L from the proximal end of the delivery member guard 2 to the proximal end of the housing 1 when the delivery member guard 2′ is in the extended position.

In a preferred example, the effective injection depth is 4 mm to 12 mm; preferably, is 6 mm, 8 mm, or 10 mm.

In one example, the delivery member guard 2′ is biased by a biasing member 4 to the proximal end of the housing 1. In a preferred example, the biasing member is a spring, e.g., a compression spring, a tension spring, a torsion spring, or a flexible arm.

In one example, the sub-assembly of the medicament delivery device comprises a drive mechanism that comprises a power source. When the power source is released, the power source is configured to act on the medicament container and force a contained medicament to be expelled via the medicament delivery member. The drive mechanism is arranged within the housing. The power source can be released only when the delivery member guard is in the retracted position. In one example, the power source is configured to be released by the delivery member guard when the delivery member guard is in the retracted position. In another example, the sub-assembly of the medicament delivery device comprises a button 3 extending out of the housing 1. In one preferred example, the button 3 extends from the distal end of the housing 1, as shown in FIG. 1. The power source is configured to be released when the button is moved towards the housing and the delivery member guard is in the retracted position.

In one example, the drive mechanism comprises a plunger rod connected to the power source. The plunger rod is configured to be moved into the medicament container by the power source when the power source is released.

In one example, the power source is a spring, a compression spring, a torsion spring, or a constant force band spring, engaged with the plunger rod. The spring is configured to move the plunger rod into the medicament container when the spring is released. Alternatively, the power source can be a gas canister containing pressurized gas or a motor.

In one example, the drive mechanism comprises a rotator rotatable around the longitudinal axis between a first position where the rotator is coupled to the plunger rod such that the plunger rod is axially immovable relative to the rotator and a second position where the rotator is decoupled from the plunger rod such that the plunger rod is axially moved by the spring. In this example, the rotator is immovable relative to the housing in the direction of the longitudinal axis L. In a preferred example, the rotator comprises a chamfered surface. The delivery member guard comprises an interacting surface configured to be moved along the chamfer surface of the rotator when the delivery member guard is moved from the extended position to the retracted position such that the rotator is moved from the first position to the second position.

In one example, the rotator comprises a tubular body extending along the longitudinal axis L. In one example, the rotator comprises a ledge extending from the tubular body of the rotator towards the longitudinal axis L. The ledge of the rotator is engaged with a proximally directed surface of the plunger rod when the rotator is in the first position, and the ledge of the rotator is disengaged from the proximally directed surface of the plunger rod when the rotator is in the second position. In another example, the housing comprises an inner housing positioned between the plunger rod and the rotator in the direction transverse to the longitudinal axis L. In this example, the inner housing comprises a flexible arm comprising a distally directed surface engaged with a proximally directed surface of the plunger rod. In this example, the tubular body of the rotator comprises a support wall configured to be aligned with the flexible arm of the inner housing when the rotator is in the first position so that the flexible arm is prevented from flexing radially outward; as a result, the distally directed surface of the flexible arm is engaged with the proximally directed surface of the plunger rod when the rotator is in the first position. The support wall of the rotator is circumferentially offset from the flexible arm when the rotator is in the second position so that the flexible arm can flex radially outward; as a result, the distally directed surface of the flexible arm is radially moved away and disengaged from the proximally directed surface of the plunger rod when the rotator is in the second position.

Alternatively, the drive mechanism comprises an actuator sleeve axially movable relative to the housing between a proximal position where the actuator sleeve is coupled to the plunger rod such that the plunger rod is axially immovable relative to the actuator sleeve and a second position where the actuator sleeve is decoupled from the plunger rod such that the plunger rod is axially moved by the power source. The actuator sleeve comprises a proximally directed surface engaged with a distally directed surface of the delivery member guard such that when the delivery member guard is moved from the extended position to the retracted position the actuator sleeve is moved from the first position to the second position by the delivery member guard.

In one example, the sub-assembly comprises an actuator positioned between the plunger rod and the actuator sleeve in the direction transverse to the longitudinal axis L. In this example, the actuator comprises a flexible arm comprising a distally directed surface engaged with a proximally directed surface of the plunger rod. In this example, the actuator sleeve comprises a support wall configured to be aligned with the flexible arm of the actuator when the actuator sleeve is in the first position so that the flexible arm is prevented from flexing radially outward; as a result, the distally directed surface of the flexible arm is engaged with the proximally directed surface of the plunger rod when the actuator sleeve is in the first position. The support wall of the actuator sleeve is axially offset from the flexible arm when the actuator sleeve is in the second position so that the flexible arm can flex radially outward. As a result, the distally directed surface of the flexible arm is radially moved away and disengaged from the proximally directed surface of the plunger rod when the actuator sleeve is in the second position.

Furthermore, in one example where the sub-assembly of the medicament delivery device comprises the button. In one example, the button is connected to the plunger rod. In this example, only when the actuator sleeve is in the second position, the plunger rod can be pressed to decouple from the actuator sleeve by being moved towards the proximal end of the housing by the button. Alternatively, in one example where the sub-assembly comprises the actuator, the button is connected to the actuator. In this example, only when the actuator is in the second position, the actuator can be pressed to decouple from the actuator sleeve by being moved towards the proximal end of the housing by the button.

In one example where the power source is a compression spring, the spring extends between the plunger rod and the actuator along the longitudinal axis L.

Furthermore, some other mechanisms that the sub-assembly of the medicament delivery device is triggered by moving the delivery member guard from the extended position to the retracted position and/or by moving the delivery member guard from the extended position to the retracted position and pressing the button towards the housing are described in commonly assigned U.S. Pat. Nos. 7,442,185, 7,597,685, US20120123350, U.S. Pat. Nos. 8,414,533, and 9,199,038 herewith, the disclosure of which are hereby incorporated by reference.

The disclosure further provides a medicament delivery device comprising a sub-assembly of the medicament delivery device as disclosed in any one of the examples above. In one example, the medicament delivery device is an autoinjector.

Furthermore, the housing, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The inventive concept has mainly been described above with reference to a few examples. However, as is readily appreciated by a person skilled in the art, other embodiments than the ones disclosed above are equally possible within the scope of the inventive concept, as defined by the appended claims.

1-12. (canceled)

13. A sub-assembly of a medicament delivery device comprising:
a housing extending along a longitudinal axis between a proximal end and a distal end, wherein the housing is configured to accommodate a medicament container, wherein the medicament container is coupled to a medicament delivery member comprising an outer part for delivering medicament, and wherein the outer part of the medicament delivery member is configured to extend beyond the proximal end of the housing in the direction of the longitudinal axis; and
a delivery member guard partially arranged within the housing, wherein the delivery member guard is telescopic relative to the proximal end of the housing, wherein the delivery member guard is movable along the longitudinal axis between an extended position where the delivery member guard is configured to fully surround the outer part of the medicament delivery member of the medicament container and a retracted position where the delivery member guard is configured to partially surround the outer part of the medicament delivery member of the medicament container, and wherein a proximal end of the delivery member guard is configured to extend beyond the proximal end of the housing when the delivery member guard is in the retracted position.

14. The sub-assembly according to claim 13, wherein when the delivery member guard is in the retracted position, a length measured along the longitudinal axis from the proximal end of the delivery member guard to the proximal end of the housing is at least 2 mm.

15. The sub-assembly according to claim 14, wherein when the delivery member guard is in the retracted position, the length measured along the longitudinal axis from the proximal end of the delivery member guard to the proximal end of the housing is between 3 mm to 6 mm.

16. The sub-assembly according to claim 15, wherein when the delivery member guard is in the retracted position, the length measured along the longitudinal axis from the proximal end of the delivery member guard to the proximal end of the housing is 3 mm, 3.3 mm, or 3.9 mm.

17. The sub-assembly according to claim 13, wherein the delivery member guard comprises a contact section configured to be in contact with a medicament delivery site, wherein the contact section comprises a contact surface perpendicular to the longitudinal axis and a neighboring sleeve extending from the contact surface in the direction of the longitudinal axis towards the distal end of the housing, and wherein the contact surface extends beyond the neighboring sleeve in the direction transverse to the longitudinal axis.

18. The sub-assembly according to claim 13, wherein the delivery member guard comprises a proximal tubular section extending beyond the proximal end of the housing when the delivery member guard is in the extended position, and wherein a length of the proximal tubular section measured along the longitudinal axis is between 14 mm to 18 mm from the proximal end of the delivery member guard to the proximal end of the housing.

19. The sub-assembly according to claim 13,
wherein the sub-assembly of the medicament delivery device comprises a drive mechanism comprises a power source, wherein when the power source is released, the power source is configured to act on the medicament container and force a contained medicament to be expelled via the medicament delivery member, wherein the drive mechanism is arranged within the housing, and
wherein the power source can be released only when the delivery member guard is in the retracted position.

20. The sub-assembly according to claim 19, wherein the power source is configured to be released by the delivery member guard when the delivery member guard is moved in the retracted position.

21. The sub-assembly according to claim 19, wherein the sub-assembly of the medicament delivery device comprises a button extending out of the housing, wherein the power source is configured to be released when the button is moved towards the housing and the delivery member guard is in the retracted position.

22. The sub-assembly according to claim 19, wherein the drive mechanism comprises a plunger rod configured to be moved into the medicament container, wherein the power source is a spring engaged with the plunger rod, and wherein the spring is configured to move the plunger rod into the medicament container when the spring is released.

23. The sub-assembly according to claim 22, wherein the drive mechanism comprises an actuator sleeve axially movable relative to the housing between a proximal position where the actuator sleeve is coupled to the plunger rod such that the plunger rod is axially immovable relative to the actuator sleeve and a second position where the actuator sleeve is decoupled from the plunger rod such that the plunger rod is axially moved by the spring, and wherein the actuator sleeve comprises a proximally directed surface engaged with a distally directed surface of the delivery member guard such that when the delivery member guard is moved from the extended position to the retracted position the actuator sleeve is moved from the first position to the second position by the delivery member guard.

24. A medicament delivery device comprising the sub-assembly according to claim 13.
